(19) United States
(12) Patent Application Publication
(10) Pub. No.: US 2021/0205495 A1
(43) Pub. Date: Jul. 8, 2021

(54) METHOD FOR MANUFACTURING SLIDING MEMBER FOR ARTIFICIAL JOINT

(71) Applicants: KYOCERA Corporation, Kyoto-shi, Kyoto (JP); The University of Tokyo, Tokyo (JP)

(72) Inventors: Shihori YAMANE, Kyoto-shi (JP); Masayuki KYOMOTO, Kyoto-shi (JP); Kazuhiko ISHIHARA, Tokyo (JP)

(73) Assignees: KYOCERA Corporation, Kyoto-shi, Kyoto (JP); The University of Tokyo, Tokyo (JP)

(21) Appl. No.: 15/998,921

(22) PCT Filed: Feb. 16, 2017
(86) PCT No.: PCT/JP2017/005789

§ 371 Date: Aug. 16, 2018

(30) Foreign Application Priority Data
Feb. 19, 2016 (JP) 2016-030427

(51) Int. Cl. A61L 27/34; A61F 2/34; A61F 2/30

(52) U.S. Cl. CPC A61L 27/34; A61F 2/34; A61L 2420/02; A61F 2002/30957; A61L 2430/24; A61F 2/30942

(57) ABSTRACT

In a molding step as step A1, a polymer material is molded and a substrate having a predetermined shape is obtained. In a polymer film forming step as step A2, the obtained substrate is immersed in a treatment aqueous solution including a compound having a phosphorylcholine group and a water-soluble inorganic salt, and the substrate in that state is irradiated with an ultraviolet light to form on a surface of the substrate a polymer film including polymer chains caused by polymerization of the compound having a phosphorylcholine group. A method for manufacturing a sliding member for an artificial joint as mentioned above makes it possible to efficiently manufacture a sliding member for an artificial joint having excellent wear resistance.

### TECHNICAL FIELD

The present invention relates to a method for manufacturing a sliding member for an artificial joint which method manufactures a sliding member preferably used for an artificial joint.

### BACKGROUND ART

There has been established a medical treatment for replacing a joint, which has lost its intrinsic function due to an injury or a disease such as osteoarthritis by a so-called artificial joint which is an artificial object having a similar function. An artificial joint is put into a living body, and therefore is required to be safe in a body and maintain a certain function over a long term. When a complication of the operation for the replacement of joint or the like occurs, there is a need to further replace the artificial joint by new one, causing an extremely large burden on the patient, and therefore such a problem must be avoided.

An artificial joint for total replacement is mainly composed of two members, and each member is fitted to an end portion of a bone. When the joint is operated, these two members relatively move to slide. In an artificial hip joint and an artificial knee joint, the members for them slide repeatedly whenever walking.

Examples of materials used in artificial joints include metals such as a cobalt-chromium alloy and a titanium alloy, ceramics such as alumina and zirconia, and polymers such as polyethylene. In an artificial hip joint, for example, a femoral head ball formed of a cobalt-chromium alloy as a femur-side member and a cup formed of polyethylene as a pelvis-side member slide, and, as these members slide repeatedly, wear particles are generated from the polyethylene cup. The generated wear particles are recognized as foreign matter in a living body, and therefore the biological immune system functions in an attempt to remove these particles. In this instance, multinucleate cells called osteoclast are activated, causing osteolysis in which the bone around the artificial joint is absorbed.

When osteolysis is caused around the artificial joint due to activation of the osteoclast, a gap is formed between the bone and the artificial joint, so that loosening of the artificial joint disadvantageously occurs.

Artificial joint members necessarily slide, and therefore have a need to use a sliding material having such excellent wear resistance that wear particles are not generated.

Patent Literature 1 discloses an artificial joint member formed of a polymer material wherein the sliding surface of the artificial joint member formed of the polymer material which is an ultra-high molecular weight polyethylene is formed of a polymer having a phosphorylcholine group.

It is known that the cartilage surface of a joint of a living body is covered with phospholipid, which contributes to protection of the cartilage bone and high lubrication. In the artificial joint member described in Patent Literature 1, the sliding surface is formed of the polymer having a phosphorylcholine group, which has a chemical structure close to that of phospholipid, and therefore excellent lubrication state is maintained for a long term, so that the artificial joint member is highly unlikely to wear and has an impact absorption function.

### CITATION LIST

### Patent Literature

Patent Literature 1: Japanese Examined Patent Publication JP-B2 4156945

### SUMMARY OF INVENTION

### Technical Problem

When a polymer film formed of a polymer having a phosphorylcholine group is graft-polymerized on the surface of an ultra-high molecular weight polyethylene material, the film needs to have a thickness of 100 nm or more for exhibiting satisfactory wear resistance properties. The conventional method described in Patent Literature 1 requires a considerably long polymerization time in order to form a film having a thickness of 100 nm or more.

For solving the above-mentioned problem, in order to increase the polymerization rate of the photoinitiated graft polymerization caused on the surface of the polyethylene on which the polymer film is formed, a method of increasing the concentration of the monomer used in the reaction or increasing the intensity of the light used for irradiation is considered.

However, the method of increasing the monomer concentration and the method of increasing the intensity of the light used for irradiation are disadvantageous in that the monomer dissolved in the solvent undergoes polymerization within the solvent, so that the resultant polymer is not bonded to the surface of the substrate. Even when the polymer is bonded to the surface of the substrate to form a 100 nm or more polymer film, the number of molecular chains in the resultant polymer per area is so small that the polymer film is easily peeled, making it difficult to obtain satisfactory wear resistance properties.

Further, there is a concern that the method of increasing the intensity of the light used for irradiation causes an adverse effect, for example, such that the total energy of the light for irradiation of the surface of the polyethylene is increased, causing the mechanical properties of the polyethylene substrate per se to become poor.

An object of the invention is to provide a method for efficiently manufacturing a sliding member for an artificial joint, which has excellent wear resistance properties.

### Solution to Problem

The invention provides a method for manufacturing a sliding member for an artificial joint, comprising:

a substrate forming step of molding a polymer material and obtaining a substrate; and

a polymer film forming step of immersing the substrate in a treatment aqueous solution comprising a compound having a phosphorylcholine group and a water-soluble inorganic salt and irradiating the substrate in that state with an ultraviolet light Lo form on at least part of a surface of the substrate a polymer film comprising polymer chains caused by polymerization of the compound having a phosphorylcholine group.

Further, in the invention, it is preferable that the water-soluble inorganic salt comprises an alkali metal salt or an alkaline earth metal salt.

Further, in the invention, it is preferable that the alkali metal salt comprises at least one selected from the group consisting of a sodium salt, a potassium salt, a lithium salt, and a cesium salt.

Further, in the invention, it is preferable that the alkaline earth metal salt comprises at least one selected from the group consisting of a calcium salt, a strontium salt, a barium salt, and a radium salt.

Further, in the invention, the phosphorylcholine-containing treatment solution containing the water-soluble inorganic salt in an amount of 0.01 to 5.0 mol/L is preferably used, the phosphorylcholine-containing treatment solution containing the water-soluble inorganic salt in an amount of 1.0 to 5.0 mol/L is more preferably used, or the phosphorylcholine-containing treatment solution containing the water-soluble inorganic salt in an amount of 1.0 to 3.0 mol/L is further preferably used.

Further, in the invention, it is preferable that the polymer film formed in the polymer film forming step has a thickness of 100 nm or more.

Further, in the invention, the time for irradiation with an ultraviolet light in the polymer film forming step is preferably 1 minute or longer, more preferably 11 to 90 minutes, or further preferably 23 to 90 minutes.

Further, in the invention, it is preferable that the polymer material comprises an ultrahigh molecular weight polyethylene material or a polyether ether ketone material. Further, the substrate may contain an additive which is at least one of, for example, an antioxidant, a crosslinker, and a reinforcer such as a carbon fiber.

Further, in the invention, it is preferable that the ultrahigh molecular weight polyethylene material has a molecular weight of one million to seven millions.

Further, in the invention, it is preferable that the polyether ether ketone material has a density of 1.2 to 1.6.

Further, in the invention, it is preferable that the substrate contains an additive which is at least one of an antioxidant, a crosslinker, and a reinforcer.

Further, in the invention, it is preferable that the polymer material comprises a crosslinking treated material.

### Advantageous Effects of Invention

According to the invention, in the substrate forming step, a polymer material is molded and a substrate is obtained, and, in the polymer film forming step, the substrate is immersed in a treatment aqueous solution containing a compound having a phosphorylcholine group and a water-soluble inorganic salt and the substrate in that state is irradiated with an ultraviolet light to form on at least part of the surface of the substrate a polymer film including polymer chains caused by polymerization of the compound having a phosphorylcholine group. By using the treatment aqueous solution containing a water-soluble inorganic salt, a polymer film having a large thickness can be formed in a short time.

Further, according to the invention, the water-soluble inorganic salt preferably comprises an alkali metal salt or an alkaline earth metal salt.

Further, according to the invention, it is preferable that the alkali metal salt comprises at least one selected from the group consisting of a sodium salt, a potassium salt, a lithium salt, and a cesium salt.

Further, according to the invention, it is preferable that the alkaline earth metal salt comprises at least one selected from the group consisting of a calcium salt, a strontium salt, a barium salt, and a radium salt.

Further, according to the invention, it is preferable that the phosphorylcholine-containing treatment solution containing the water-soluble inorganic salt in an amount of 0.01 to 5.0 mol/L is used.

Further, according to the invention, it is preferable that the phosphorylcholine-containing treatment solution containing the water-soluble inorganic salt in an amount of 1.0 to 5.0 mol/L is used.

Further, according to the invention, it is preferable that the phosphorylcholine-containing treatment solution containing the water-soluble inorganic salt in an amount of 1.0 to 3.0 mol/L is used.

Further, according to the invention, the polymer film formed in the polymer film forming step has a thickness of 100 nm or more, and an extremely large film thickness can be obtained.

Further, according to the invention, the time for irradiation with an ultraviolet light is 1 minute or longer, and a satisfactory polymer film can be obtained in an extremely short time.

Further, according to the invention, the time for irradiation with an ultraviolet light is preferably 11 to 90 minutes, and a satisfactory polymer film can be obtained in a shorter time than the time for the existing methods.

Further, according to the invention, the time for irradiation with an ultraviolet light is more preferably 23 to 90 minutes, and a satisfactory polymer film can be obtained in a shorter time than the time for the existing methods.

Further, according to the invention, it is preferable that the polymer material comprises an ultra-high molecular weight polyethylene material or a polyether ether ketone material.

Further, according to the invention, it is preferable that the ultra-high molecular weight polyethylene material has a molecular weight of one million to seven millions.

Further, according to the invention, it is preferable that the polyether ether ketone material has a density of 1.2 to 1.6.

Further, according to the invention, the substrate may contain an additive such as an antioxidant, a crosslinker, or a reinforcer.

Further, according to the invention, it is preferable that the polymer material comprises a crosslinking treated material.

### BRIEF DESCRIPTION OF DRAWINGS

Other and further objects, features, and advantages of the invention will be more explicit from the following detailed description taken with reference to the drawings wherein:

FIG. 1 is a flow sheet showing a method for manufacturing a sliding member for an artificial joint according to a first embodiment of the invention;

FIG. 2 is a flow sheet showing a method for manufacturing a sliding member for an artificial joint according to a second embodiment of the invention;

FIG. 3 is a diagrammatic view of an artificial hip joint 1 which is one kind of artificial joint;

FIG. 4 is a diagrammatic view of an acetabular cup 10;

FIG. 5A is a graph showing a change of the polymer film property with the ultraviolet light irradiation time and the sodium chloride concentration, and FIG. 5A is a graph showing a change of the static contact angle of water;

FIG. 5B is a graph showing a change of the phosphoric acid index with the ultraviolet light irradiation time and the sodium chloride concentration;

FIG. 5C is a graph showing a change of the film thickness with the ultraviolet light irradiation time and the sodium chloride concentration; and

FIG. 5D is a graph showing a change of the coefficient of friction with the ultraviolet light irradiation time and the sodium chloride concentration.

### DESCRIPTION OF EMBODIMENTS

Hereinbelow, a preferred embodiment of the invention will be described in detail with reference to the drawings.

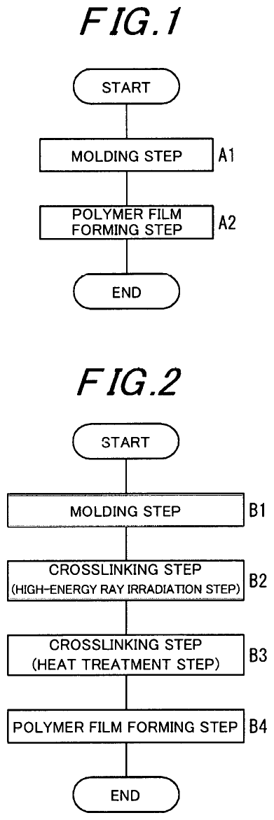

FIG. 1 is a flow sheet showing a method for manufacturing a sliding member for an artificial joint according to the first embodiment of the invention.

The method of the first embodiment comprises the following two steps:

(step A1) a molding step, and

(step A2) a polymer film forming step.

In the molding step which is the step A1, a polymer material is molded and a substrate having a predetermined shape is obtained. In the present embodiment, the substrate forming step comprises the molding step as the step A1.

As a polymer material constituting the substrate, in the invention, an ultra-high molecular weight polyethylene (UHMWPE) material can be used. The UHMWPE is excellent in mechanical properties, such as a wear resistance, an impact resistance, and a deformation resistance, and is advantageously used as a resin material for use in an artificial joint. The higher the molecular weight, the higher the wear resistance, and the molecular weight is preferably one million or more, more preferably one million to seven millions, or more preferably three millions to four millions. The molecular weight of the UHMWPE constituting the substrate is determined from the expression (1) below by the measurement of a viscosity of a decahydronaphthalene (decalin) solution at 135° C.

Molecular weight=5.37×10⁴×(Intrinsic viscosity)^{1.49} (1)

Further, as a polymer material constituting the substrate, in the invention, a polyether ether ketone (PEEK) material can be used. The PEEK is excellent in mechanical properties such as an impact resistance and a deformation resistance, and is advantageously used as a resin material for use in an artificial joint.

The substrate is obtained by charging UHMWPE or PEEK in a powdery form, a particulate form, or a pellet form into a mold and subjecting it to compression molding, extrusion molding, or injection molding. The UHMWPE or PEEK is a thermoplastic resin, but has low fluidity even at the melting temperature thereof or higher, and therefore UHMWPE or PEEK in a solid state is advantageously charged into a mold and subjected to molding under conditions at a high temperature and at a high pressure.

The substrate may be subjected to molding after adding thereto, for example, an antioxidant, a crosslinker, or a reinforcer such as a carbon fiber.

The substrate obtained by compression molding or extrusion molding may be subjected as such to the subsequent polymer film forming step, or may be subjected to cutting so as to have an appropriate shape and then subjected to the polymer film forming step.

Next, in the polymer film forming step as the step A2, the obtained substrate is immersed in an aqueous solution containing a polymerizable monomer, which is a compound having a phosphorylcholine group (PC compound), and a water-soluble inorganic salt and the substrate in that state is irradiated with an ultraviolet light to form on the surface of the substrate a polymer film including polymer chains caused by polymerization of the PC compound.

The polymer film is formed in order to reduce the coefficient of friction of the sliding surface of the substrate, and therefore may be formed on at least a portion corresponding to the sliding surface which is part of the surface of the substrate. For example, when an acetabular cup of an artificial hip joint is manufactured, the polymer film may be formed on at least the spherical surface in the cup where the femoral head ball slides.

The formation of the polymer film on the surface of the substrate is made by photoinitiated graft polymerization of polymer chains caused by polymerization of the PC compound on the surface corresponding to the sliding surface of the substrate formed from a polymer.

The photoinitiated graft polymerization can cause the polymer chains of the PC compound to be stably fixed to the surface of the substrate. Further, the photoinitiated graft polymerization can form a phosphorylcholine group in a large amount on the sliding surface of the substrate to increase the density of the polymer film.

A polymerizable monomer which is the PC compound is used for forming the polymer film, and particularly, by selecting a polymerizable monomer having a phosphorylcholine group at one end and a functional group graft-polymerizable on the polymer substrate at another end, it is possible to obtain a polymer film grafted to the sliding surface of the substrate.

Examples of polymerizable monomers used in the embodiment of the invention include 2-methacryloyloxyethyl phosphorylcholine, 2-acryloyloxyethyl phosphorylcholine, 4-methacryloyloxybutyl phosphorylcholine, 6-methacryloyloxyhexyl phosphorylcholine, ω-methacryloyloxyethylene phosphorylcholine, and 4-styryloxybutyl phosphorylcholine. Of these, especially preferred is 2-methacryloyloxyethyl phosphorylcholine (hereinafter, referred to as “MPC”).

MPC has a chemical structure represented by the structural formula shown below, and is a polymerizable monomer having a phosphorylcholine group and polymerizable methacrylate units. MPC has a feature such that radical polymerization can easily cause MPC to suffer polymerization, forming a high molecular-weight homopolymer (Ishihara et al.: Polymer Journal, vol. 22, page 355 (1990)). Therefore, when a polymer film is formed in an aggregate form of polymer chains caused by polymerization of MPC, the MPC polymer chains can be grafted to the sliding surface of the substrate under relatively mild conditions, and further a polymer film having a high density is formed, so that a phosphorylcholine group in a large amount can be formed on the sliding surface of the substrate.

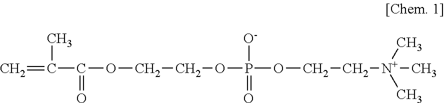 [SMILES: C=C(C)C(=O)OCCOP(=O)([O-])OCC[N+](C)(C)C]

The polymer film formed in the present embodiment can be not only a homopolymer comprising a single polymerizable monomer having a phosphorylcholine group, but also a copolymer comprising a polymerizable monomer having a phosphorylcholine group and, for example, another vinyl compound monomer. By appropriately selecting the type of the another vinyl compound used, a function of improving the mechanical strength or the like can be imparted to the polymer film.

The water-soluble inorganic salt used in the embodiment of the invention is an alkali metal salt or alkaline earth metal salt. The alkali metal salt comprises at least one selected from the group consisting of a sodium salt, a potassium salt, a lithium salt, and a cesium salt. The alkaline earth metal salt comprises at least one selected from the group consisting of a calcium salt, a strontium salt, a barium salt, and a radium salt.

In the embodiment of the invention, by using the water-soluble inorganic salt, the formation of a polymer film having a thickness of 100 nm or more on the surface of the substrate in a period of time as short as 1 to 90 minutes has been realized.

Further, in the embodiment of the invention, by using the water-soluble inorganic salt, there has been realized the formation in a short time of a polymer film having a thickness of 600 nm or more which has not conventionally been expected at all. Even in the film having such a large thickness, a gap or the like is not observed in the interface between the film and the substrate, and the film is expected to have a satisfactory film strength.

For obtaining a polymer film grafted to the sliding surface of the substrate, a photopolymerization initiator is applied to the sliding surface of the substrate, and the resultant substrate is immersed in an aqueous solution containing the PC compound, which is a polymerizable monomer, and a water-soluble inorganic salt (hereinafter, referred to as “polymerization treatment solution”), and the sliding surface of the substrate in that state is irradiated with an ultraviolet light (for example, having a wavelength of 300 to 400 nm). When the sliding surface of the substrate is irradiated with an ultraviolet light, the PC compound near the sliding surface undergoes polymerization to form polymer chains, and the formed polymer chains are grafted to the polymer substrate which is the sliding surface. The polymer chains are grafted to the sliding surface with a high density, forming a polymer film which coats the whole of the sliding surface of the substrate.

For example, for obtaining a polymer film grafted to the sliding surface of a substrate containing a photopolymerization initiator group, such as PEEK, the substrate is immersed in a polymerization treatment solution, and the sliding surface of the substrate in that state is irradiated with an ultraviolet light (for example, having a wavelength of 300 to 400 nm). When the sliding surface of the substrate is irradiated with an ultraviolet light, the PC compound near the sliding surface undergoes polymerization to form polymer chains, and the formed polymer chains are grafted to the polymer substrate which is the sliding surface. The polymer chains are grafted to the sliding surface with a high density, forming a polymer film which coats the whole of the sliding surface of the substrate.

As a light source for the ultraviolet light irradiation, for example, a high-pressure mercury lamp (UVL-400HA, manufactured by Riko-Kagaku Sangyo Co., Ltd.), an LED (MeV365-P601JMM, manufactured by Yen electron Volt Co., Ltd.) or the like can be used.

In the present embodiment, grafting using a photopolymerization initiator is made, and ultraviolet light irradiation causes photopolymerization initiator radicals to be generated, and the generated photopolymerization initiator radicals form polymerization initiating sites on the surface of the substrate, and the ends of polymer chains of the PC compound which is a polymerizable monomer react with the polymerization initiating sites to cause bonding of a branched polymer and the subsequent growth of the branched polymer.

In the polymer film forming step, the water-soluble inorganic salt concentration of the polymerization treatment solution is 0.01 to 5.0 mol/L. Further, the water-soluble inorganic salt concentration is preferably in the range of from 1.0 to 5.0 mol/L, or more preferably in the range of from 1.0 to 3.0 mol/L.

In the polymer film forming step, an ultraviolet light irradiation time is 1 minute or longer. Further, the ultraviolet light irradiation time is preferably in the range of from 11 to 90 minutes, or more preferably in the range of from 23 to 90 minutes. Further, it is preferable that, after the polymer film forming step, a sterilization treatment by gamma ray irradiation is performed.

Thus, a sliding member for an artificial joint having the surface of the substrate coated with the polymer film is obtained.

FIG. 2 is a flow sheet showing a method for manufacturing a sliding member for an artificial joint according to a second embodiment of the invention.

The method of the second embodiment comprises the following four steps:

(step B1) a molding step,

(step B2) a crosslinking step (high-energy ray irradiation step),

(step B3) a crosslinking step (heat treatment step), and

(step B4) a polymer film forming step.

In the present embodiment, the substrate forming step comprises a molding step which is the step B1, and crosslinking steps which are the steps B2 and B3. The steps B2 and B3 can be omitted.

The molding step as the step B1 is the same as the molding step which is the step A1 in the first embodiment, and the polymer film forming step as the step B4 is the same as the polymer film forming step which is the step A2 in the first embodiment, and therefore the detailed descriptions thereof are omitted in the present embodiment.

In the crosslinking step (high-energy ray irradiation step) as the step B2, a substrate formed of UHMWPE is irradiated with a high-energy ray, for example, an X-ray, a gamma ray, or an electron beam, to generate free radicals so that molecular chains of UHMWPE are bonded to each other, forming a network structure (crosslinked, CL) of UHMWPE. A crosslinked structure is formed in the molecule, so that mechanical properties, such as a wear resistance and an impact resistance, are improved.

The crosslinking reaction can be caused by adding a crosslinker, but the unreacted crosslinker cannot be completely removed, and therefore, taking the effect of the unreacted crosslinker on a living body into consideration, a crosslinking reaction caused by high-energy ray irradiation is preferable. In the present embodiment, the irradiation dose of a high-energy ray is 25 to 150 kGy.

As a high-energy ray source, for example, as a gamma ray source, a radiation apparatus using Co (cobalt) 60 as a radiation source, an accelerator for emitting an electron beam, an apparatus for emitting an X-ray, or the like can be used.

In the crosslinking step (heat treatment step) as the step B3, free radicals generated by the high-energy ray irradiation in the step B2 are more efficiently consumed in the crosslinking reaction to accelerate the intramolecular crosslinking. The temperature for the heat treatment is preferably in the range of from 110 to 130° C., and the treatment time for the heat treatment is preferably in the range of from 2 to 12 hours.

In the second embodiment, the crosslinking step causes a crosslinked structure in the molecule, obtaining a substrate having further improved mechanical properties such as a wear resistance and an impact resistance.

The obtained substrate is subjected to photoinitiated graft polymerization under the same reaction conditions as those in the first embodiment so that the sliding surface of the substrate is coated with a polymer film. Thus, a sliding member for an artificial joint having further improved properties is obtained.

In applying the sliding member for an artificial joint manufactured by the method of the invention to an artificial joint, both the femoral head and the acetabulum may be replaced by the artificial joint member in the invention, and one of the femoral head and the acetabulum may be replaced by the artificial joint member in the invention, and the remaining one may be replaced by a member formed of, for example, a metal such as stainless steel or a cobalt-chromium alloy, ceramics such as alumina or zirconia, or a polymer such as UHMWPE or PEEK. Alternatively, there may be employed an artificial joint member of the femoral head and/or the acetabulum of a composite form in which the artificial joint member in the invention constitutes only the sliding portion and another polymer material or the above metal, ceramics or the like constitutes the other portion.

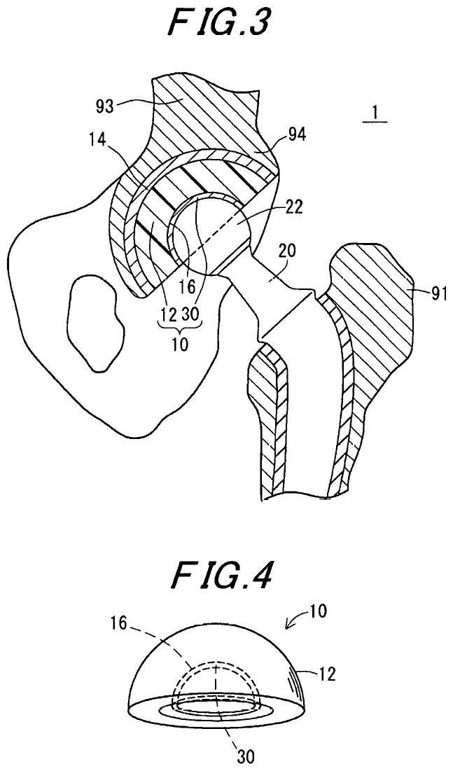

FIG. 3 is a diagrammatic view of an artificial hip joint 1 which is an artificial joint, and FIG. 4 is a diagrammatic view of an acetabular cup 10. The artificial hip joint 1 comprises an acetabular cup 10 fixed to an acetabulum 94 of a coxal bone 93, and a femoral stem 20 fixed to the proximal end of a femur 91. The acetabular cup 10 has a cup substrate 12 having an acetabulum fixing surface 14 (outer diameter) which is almost hemispherical and a sliding surface 16 (inner diameter) which is almost hemispherically depressed, and a polymer film 30 coating the inside sliding surface 16. A femoral head 22 of the femoral stem 20 is inserted into the depression of the acetabular cup 10 on which the polymer film 30 is formed, and slides to function as a hip joint.

The acetabular cup 10 is composed of a sliding member manufactured by the above-mentioned first and second embodiments. As shown in FIG. 4, in the acetabular cup 10 manufactured by the embodiment of the invention, the sliding surface 16 inside the cup substrate 12 is coated with the polymer film 30, and the polymer film 30 is obtained by graft polymerization of polymer chains having a phosphorylcholine group on the sliding surface 16.

The polymer film 30 has a structure similar to the structure of a biological film, and has high affinity with the lubricating liquid present at a joint and can keep the lubricating liquid inside the film, and therefore can reduce the coefficient of friction, as compared to the bare sliding surface 16 in the conventional acetabular cup 10.

Thus, the acetabular cup 10 is obtained as a member having the sliding properties improved and having the wear resistance improved.

### Embodiment 1

Hereinbelow, studies are made on the relationship between the concentration of water-soluble inorganic salt contained in the treatment aqueous solution in which a substrate formed from UHMWPE is immersed, and the properties of the polymer film formed on the surface of the substrate.

The properties of the formed polymer film were evaluated in terms of (a) a contact angle of water, (b) a phosphoric acid index, (c) a film thickness, and (d) a coefficient of friction. Test specimens for conducting the evaluation were prepared as follows.

A polymer film including polymer chains caused by polymerization of the compound having a phosphorylcholine group was formed on one of the 10 mm×100 mm planes among the 6 planes of a square bar (section: 10 mm×3 mm; length: 100 mm) formed of UHMWPE having a molecular weight of three millions to four millions.

A square bar formed of UHMWPE was first immersed in an acetone solution containing benzophenone (1.0 g/dL) for 30 seconds, and then quickly withdrawn from the solution and the solvent was removed at room temperature to absorb benzophenone on the square bar.

Polymerization treatment solutions having an MPC content of 0.5 mol/L and having different contents of sodium chloride which is a water-soluble inorganic salt, i.e., having sodium chloride contents of 0 mol/L, 0.5 mol/L, 1.0 mol/L, 1.5 mol/L, 2.0 mol/L, 2.5 mol/L, and 3.0 mol/L, respectively, were individually preliminarily kept at 60° C., and then satisfactorily deaerated. The UHMWPE square bar on which benzophenone had satisfactorily adsorbed was immersed in each polymerization treatment solution, and the square bar was irradiated with an ultraviolet light having a wavelength of 300 to 400 nm and an intensity of 5 mW/cm² for 11 minutes, 23 minutes, 45 minutes, and 90 minutes, respectively, and withdrawn from the polymerization treatment solution and then washed well with pure water and ethanol to obtain a test specimen having a polymer film formed from poly(MPC) (hereinafter, referred to as “PMPC”). The specimens in which the sodium chloride contents are 0 mol/L, 0.5 mol/L, 1.0 mol/L, 1.5 mol/L, 2.0 mol/L, 2.5 mol/L, and 3.0 mol/L, respectively, correspond to Examples of the invention.

(a) Contact Angle of Water

It is considered that when the hydrophilicity of the polymer film 30 which coats the sliding surface 16 of the acetabular cup 10 is high, the acetabular cup has affinity with the lubricating liquid in a living body. The polymer film 30 which is satisfactorily wet due to the lubricating liquid is expected to impart high lubrication properties to the acetabular cup 10, making it possible to enhance the durability of the acetabular cup 10.

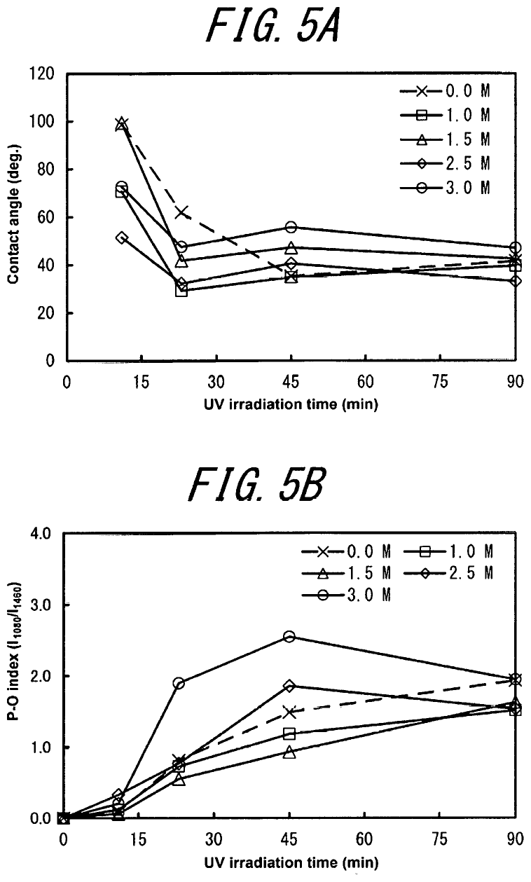

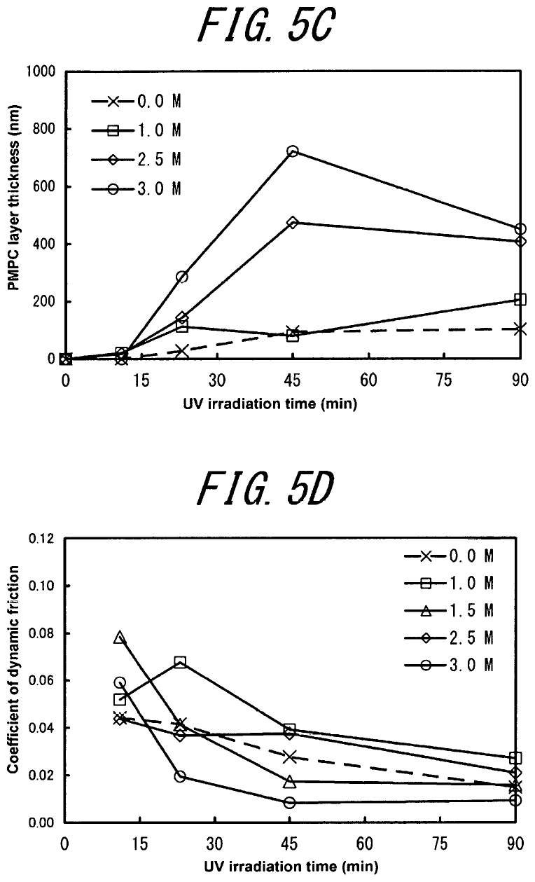

The hydrophilicity of the polymer film 30 was evaluated by measuring a contact angle of a droplet of pure water placed on the surface of each test specimen on which the polymer film is formed. A static contact angle of water was evaluated by a sessile drop method using a surface contact angle measurement apparatus (DM300, manufactured by Kyowa Interface Science Co., Ltd.). The measurement of a static surface contact angle by a sessile drop method was conducted in accordance with the ISO 15989 standards in which 1 μL of a droplet of pure water is placed on the surface of a specimen and, after a time of 60 seconds has lapsed, a contact angle of the water is measured. The results are shown in the graph of FIG. 5A.

(b) Phosphoric Acid Index

The polymer film 30 is obtained from polymer chains which are caused by polymerization of the PC compound and bonded to the sliding surface 16 of the cup substrate 12, and it is considered that the higher the “density” of the polymer film 30 is, the higher the affinity with the lubricating liquid is, and properties such as a wear resistance are improved.

A “density” of the polymer film is an amount of the polymer chains of the PC compound present on the surface of the substrate per unit area, and can be used as an index indicating how close the polymer chains caused by polymerization of the PC compound present per unit area are to one another. Therefore, when the density is higher, the polymer chains caused by polymerization of the PC compound present on the surface of the substrate can be considered to be more close to one another.

As an index for the density of the polymer film, a “phosphoric acid index” was introduced to quantitatively evaluate the degree of closeness of the PC compound.

The “phosphoric acid index” is defined as I₁₀₈₀/I₁₄₆₀ that is the intensity ratio of the peak intensity I₁₀₈₀ at 1080 cm⁻¹ which is an absorption of a phosphoric acid group, to the peak intensity I₁₄₆₀ at 1460 cm⁻¹ which is an absorption of a methylene group, in a spectrum obtained by a Fourier transform infrared spectroscopy (FT-IR) analysis.

When a polymer film containing the PC compound is formed on a substrate formed of UHMWPE having a methylene group and subjected to FT-IR measurement as conducted in the invention, a peak ascribed to a methylene group derived from the substrate and a peak ascribed to a phosphoric acid group derived from the polymer film are observed. In this instance, if the composition of the substrate is constant and the thickness of the polymer film does not change extremely (for example, a difference in the thickness is within 1 μm), a phosphoric acid index determined from the peak intensity of an absorption of a methylene group and the peak intensity of an absorption of a phosphoric acid group is almost proportional to the number of phosphoric acid groups present on the surface of the substrate per unit area.

The FT-IR measurement was performed using an FT-IR apparatus (FT/IR-6300 type A, manufactured by JASCO Corporation) at a resolution of 4 cm⁻¹ with an accumulation frequency of 64. The results are shown in the graph of FIG. 5B.

(c) Film Thickness

When the surface of the substrate can be coated with the polymer film 30 having a larger thickness in the state of being in close contact with the substrate, the durability of the acetabular cup 10 is improved.

A thickness of the polymer film was measured by embedding a specimen used for the measurement in an epoxy resin and dyeing it with ruthenium tetrachloride and then, cutting an ultrathin section out of the specimen using an ultramicrotome and observing the cut surface using a transmission electron microscope (JEM-1010 type, manufactured by JEOL LTD.) at an accelerating voltage of 100 kV. With respect to the obtained electron microscope images, a film thickness in the cut surface was measured at 10 points per image, and an arithmetic average of the thickness values was determined and used as a film thickness. The results are shown in the graph of FIG. 5C.

(d) Coefficient of Friction

When the coefficient of friction of the polymer film 30 which coats the sliding surface 16 of the acetabular cup 10 is small, it is expected that the abrasion wear of the polymer film 30 due to sliding of the acetabular cup with the femoral head 22 can be suppressed, making it possible to improve the durability of the acetabular cup 10.

Using pure water as a lubricating liquid, a spherical specimen made of a cobalt-chromium alloy having a diameter of 9 mm was pressed to vertically apply a load of 0.98 N to the contact surface of the spherical metal specimen with the surface of a test specimen on which the polymer film is formed, and then only the spherical specimen was allowed to slide at a speed of 3000 mm/min on the surface of the square bar, determining a coefficient of dynamic friction. In the measurement of a coefficient of dynamic friction, a ball-on-plate type friction testing apparatus was used. The results are shown in the graph of FIG. 5D.

FIGS. 5A to 5D are graphs showing changes of the polymer film properties with the ultraviolet light irradiation time and the sodium chloride concentration. FIG. 5A is a graph showing a change of the contact angle of water, wherein the abscissa indicates an ultraviolet light irradiation time (minute) [UV irradiation time (min)], and the ordinate indicates a static contact angle)(° of water [Contact angle (deg.)]. FIG. 5B is a graph showing a change of the phosphoric acid index, wherein the abscissa indicates an ultraviolet light irradiation time (minute), and the ordinate indicates a phosphoric acid index I₁₀₈₀/I₁₄₆₀ (−) [P—O index (I₁₀₈₀/I₁₄₆₀)]. FIG. 5C is a graph showing a change of the film thickness, wherein the abscissa indicates an ultraviolet light irradiation time (minute), and the ordinate indicates a film thickness (nm) [PMPC layer thickness (nm)]. FIG. 5D is a graph showing a change of the coefficient of dynamic friction, wherein the abscissa indicates an ultraviolet light irradiation time (minute), and the ordinate indicates a coefficient of dynamic friction (−) [Coefficient of dynamic friction].

As shown in the graph of FIG. 5A, when the ultraviolet light irradiation time is from 11 to 23 minutes, the contact angle is reduced, which indicates that the hydrophilicity becomes higher, and the contact angle is not markedly changed thereafter. Further, the contact angle is not largely affected by the sodium chloride concentration.

As shown in the graph of FIG. 5B, in the case where the sodium chloride concentration is low (1.0 to 1.5 mol/L), the phosphoric acid index is increased as the ultraviolet light irradiation time is increased. In the case where the sodium chloride concentration is high (2.5 to 3.5 mol/L), the phosphoric acid index is markedly increased when the ultraviolet light irradiation time is from 11 to 45 minutes, and the phosphoric acid index is slightly reduced when the ultraviolet light irradiation time is 90 minutes.

As shown in the graph of FIG. 5C, the film thickness is largely affected by the sodium chloride concentration. In the case where the sodium chloride concentration is 0 mol/L, the film thickness is 103 nm even when the ultraviolet light irradiation time is 90 minutes. By contrast, in the case where the sodium chloride concentration is 1.0 mol/L, the film thickness is 205 nm when the ultraviolet light irradiation time is 90 minutes, in the case where the sodium chloride concentration is 2.5 mol/L, the film thickness is 474 nm when the ultraviolet light irradiation time is 45 minutes, and, in the case where the sodium chloride concentration is 3.0 mol/L, the film thickness is 721 nm when the ultraviolet light irradiation time is 45 minutes. Such a large film thickness cannot be achieved by a conventional method using no water-soluble inorganic salt, and an increase of the thickness of the polymer film by the addition of a water-soluble inorganic salt to the polymerization treatment solution is a remarkable effect.

As shown in the graph of FIG. 5D, when the ultraviolet light irradiation time is from 11 to 23 minutes, the coefficient of friction is markedly reduced, and the coefficient of friction is not markedly changed thereafter. Further, a tendency is seen such that the higher the sodium chloride concentration is, the lower the coefficient of friction is.

The contact angle and coefficient of friction are properties of the surface of the polymer film, and it shows that the surface properties are equivalent even when the film thicknesses are different.

### Embodiment 2

Hereinbelow, studies are made, using a PEEK substrate instead of the substrate formed of UHMWPE, on the relationship between the concentration of the PC compound, which is a polymerizable monomer contained in the treatment aqueous solution in which the PEEK substrate is immersed, and the properties of the polymer film formed on the surface of the PEEK substrate.

The properties of the formed polymer film were evaluated in terms of (a) a contact angle of water, (b) a phosphoric acid index, (c) a film thickness, and (d) a coefficient of friction. Test specimens for conducting the evaluation were prepared as follows.

A polymer film including polymer chains caused by polymerization of the compound having a phosphorylcholine group was formed on a sheet material formed of PEEK (area: 10 mm×10 mm; thickness: 0.5 mm) having a density of 1.3 to 1.4.

Polymerization treatment solutions having an MPC content of 0.5 mol/L and having different contents of sodium chloride which is a water-soluble inorganic salt, i.e., having a sodium chloride content of 0 mol/L, 0.5 mol/L, 1.0 mol/L, 1.5 mol/L, 2.0 mol/L, 2.5 mol/L, 3.0 mol/L, 3.5 mol/L, and 4.0 mol/L, respectively, were individually preliminarily kept at 60° C., and then satisfactorily deaerated. The PEEK sheet material was immersed in each polymerization treatment solution, and the PEEK sheet material was irradiated with an ultraviolet light having a wavelength of 300 to 400 nm and an intensity of 20 mW/cm² for 5 minutes, and withdrawn from the polymerization treatment solution and then washed well with pure water and ethanol to obtain a test specimen having a polymer film formed from PMPC. The specimens having a polymer film formed on the surface of the PEEK substrate, in which the sodium chloride contents are 0 mol/L, 0.5 mol/L, 1.0 mol/L, 1.5 mol/L, 2.0 mol/L, 2.5 mol/L, 3.0 mol/L, and 4.0 mol/L, correspond to Examples of the invention, respectively.

(a) Contact Angle of Water

In accordance with the same method as described in Embodiment 1, the hydrophilicity of the polymer film formed on the surface of the PEEK substrate was evaluated by measuring a contact angle of a droplet of pure water placed on the surface of each test specimen on which the polymer film is formed. With respect to all the conditions for the polymerization of the polymer film, the static contact angle of water on the surface of the PEEK substrate on which the polymer film is formed was about 20°. In contrast, the static contact angle of water on the surface of the untreated PEEK substrate was about 70°.

(b) Film Thickness

A thickness of the polymer film was measured by means of a spectroscopic ellipsometer (Lincoln type, manufactured by J. A. Woollam Co., Inc.). The measurement was performed at an incident angle of 70°, and a film thickness was determined by making a calculation using a Cauchy model and using a refractive index of 1.49 at a wavelength of 632.8 nm.

With respect to the polymer film formed on the surface of the PEEK substrate under conditions such that the ultraviolet light irradiation time is 5 minutes, the film thickness was increased in proportion to the sodium chloride content, and, in the case where the sodium chloride content is 1.0 mol/L or more, a polymer film having a thickness equivalent to the thickness of the polymer film formed by a conventional method for a period of time of 90 minutes was formed. The formation of a polymer film having such a satisfactory thickness in a period of time as short as 5 minutes cannot be achieved by a conventional method using no water-soluble inorganic salt, and an increase of the thickness of the polymer film by the addition of a water-soluble inorganic salt to the polymerization treatment solution is a remarkable effect.

The invention may be embodied in other specific forms without departing from the spirit or essential characteristics thereof. The present embodiments are therefore to be considered in all respects as illustrative and not restrictive, the scope of the invention being indicated by the appended claims rather than by the foregoing description and all changes which come within the meaning and the range of equivalency of the claims are therefore intended to be embraced therein.

### REFERENCE SIGNS LIST

1: Artificial hip joint
10: Acetabular cup
12: Cup substrate
14: Acetabulum fixing surface
16: Sliding surface
20: Femoral stem
22: Femoral head
30: Polymer film
91: Femur
93: Coxal bone
94: Acetabulum

1. A method for manufacturing a sliding member for an artificial joint, comprising:
a substrate forming step of molding a polymer material and obtaining a substrate; and
a polymer film forming step of immersing the substrate in a treatment aqueous solution comprising a compound having a phosphorylcholine group and a water-soluble inorganic salt, and irradiating the substrate in that state with an ultraviolet light to form on at least part of a surface of the substrate a polymer film comprising polymer chains caused by polymerization of the compound having a phosphorylcholine group.

2. The method for manufacturing a sliding member for an artificial joint according to claim 1, wherein the water-soluble inorganic salt comprises an alkali metal salt or an alkaline earth metal salt.

3. The method for manufacturing a sliding member for an artificial joint according to claim 2, wherein the alkali metal salt comprises at least one selected from the group consisting of a sodium salt, a potassium salt, a lithium salt, and a cesium salt.

4. The method for manufacturing a sliding member for an artificial joint according to claim 2, wherein the alkaline earth metal salt comprises at least one selected from the group consisting of a calcium salt, a strontium salt, a barium salt, and a radium salt.

5. The method for manufacturing a sliding member for an artificial joint according to claim 1, wherein a concentration of the water-soluble inorganic salt is 0.01 to 5.0 mol/L.

6. The method for manufacturing a sliding member for an artificial joint according to claim 1, wherein a concentration of the water-soluble inorganic salt is 1.0 to 5.0 mol/L.

7. The method for manufacturing a sliding member for an artificial joint according to claim 1, wherein a concentration of the water-soluble inorganic salt is 1.0 to 3.0 mol/L.

8. The method for manufacturing a sliding member for an artificial joint according to claim 1, wherein the polymer film formed in the polymer film forming step has a thickness of 100 nm or more.

9. The method for manufacturing a sliding member for an artificial joint according to claim 1, wherein an ultraviolet light irradiation time in the polymer film forming step is 1 minute or longer.

10. The method for manufacturing a sliding member for an artificial joint according to claim 1, wherein an ultraviolet light irradiation time in the polymer film forming step is 11 to 90 minutes.

11. The method for manufacturing a sliding member for an artificial joint according to claim 1, wherein an ultraviolet light irradiation time in the polymer film forming step is 23 to 90 minutes.

12. The method for manufacturing a sliding member for an artificial joint according to claim 1, wherein the polymer material comprises an ultra-high molecular weight polyethylene material or a polyether ether ketone material.

13. The method for manufacturing a sliding member for an artificial joint according to claim 12, wherein the ultrahigh molecular weight polyethylene material has a molecular weight of one million to seven millions.

14. The method for manufacturing a sliding member for an artificial joint according to claim 12, wherein the polyether ether ketone material has a density of 1.2 to 1.6.

15. The method for manufacturing a sliding member for an artificial joint according to claim 1, wherein the substrate comprises an additive which is at least one of an antioxidant, a crosslinker, and a reinforcer.

16. The method for manufacturing a sliding member for an artificial joint according to claim 1, wherein the polymer material comprises a crosslinking treated material.
